(19) United States
(12) Patent Application Publication
(10) Pub. No.: US 2005/0267384 A1
(43) Pub. Date: Dec. 1, 2005

(54) Blood collection kit adapter

(76) Inventors: Kevin Paul Sauer, Richmond, CA (US); Jeffrey Lynn Hancock, Lakeport, CA (US)

Correspondence Address: BANNER & WITCOFF, LTD., 1001 G STREET, N.W., WASHINGTON, DC, 20001-4597, US

(21) Appl. No.: 11/136,819

(22) Filed: May 24, 2005

Related U.S. Application Data
(60) Provisional application No. 60/576,099, filed on Jun. 1, 2004.

(51) Int. Cl. A61B 5/00; B65D 81/00

(52) U.S. Cl. 600/577; 600/576

(57) ABSTRACT

An adapter for collecting a blood sample is disclosed that includes a stopper and a housing. The stopper is formed of a material that is piercable by a needle and sealable upon withdrawal of the needle. The housing, which has a first end and an opposite second end, defines an aperture extending through the housing from the first end to the second end. The stopper is located in the aperture and adjacent the first end. In addition, the second end has a configuration that is joinable with a syringe. The adapter may be a part of a kit that includes a needle device, a holder, and a syringe, for example. A method of collecting a first blood sample and a second blood sample is also disclosed.

### CROSS-REFERENCE TO RELATED APPLICATION

This non-provisional U.S. patent application claims priority to provisional U.S. patent application Ser. No. 60/576,099, which was filed in the U.S. Patent and Trademark Office on Jun. 1, 2004 and entitled Blood Collection Kit Adapter, such provisional U.S. patent application being entirely incorporated herein by reference.

### BACKGROUND OF THE INVENTION

1. Field of the Invention

The present invention relates to devices for collecting blood samples. The invention concerns, more particularly, an adapter for use in collecting blood in order to gain general blood data as well as blood gas data. The adapter has application to blood draw kits utilized in the medical as well as veterinary technologies. The invention also concerns a method of collecting one or more blood samples.

2. Description of Background Art

Phlebotomists routinely collect blood samples from individuals at the direction of physicians or other healthcare professionals. The blood samples are analyzed in a laboratory for purposes of diagnosing illness or determining whether various blood component parameters are within an acceptable range. In general, two types of blood samples are collected by phlebotomists: venous blood samples and arterial blood samples. Whereas a venous blood sample is collected by drawing blood from a vein of an individual, an arterial blood sample is collected by drawing blood from an artery of the individual.

The type of blood sample (i.e., venous or arterial) collected by the phlebotomist depends upon the information that the physician intends to gain from the blood sample. Venous blood samples are generally utilized to provide the physician with information on, for example, (a) the blood cell count; (b) the level of electrolytes and metabolytes in the blood; (c) the presence of various enzymes and proteins; (d) the level of ions and trace metals; (e) the presence of lipids, such as triglycerides and cholesterol; and (f) the existence of tumor indicators, bacteria, or viral agents. For purposes of reference in the following material, the information that may be gained through a venous blood sample will be referred to as general blood data. Arterial blood samples, on the other hand, are utilized when information regarding the ability of the lungs to exchange carbon dioxide for oxygen is required by the physician. More particularly, arterial blood samples are utilized to determine the relative percentage and quantity of blood gasses, such as oxygen and carbon dioxide, that are present in the blood after passing through lungs of the individual. For purposes of reference in the following material, this information will be referred to as blood gas data.

A blood sample utilized to gain blood gas data is taken from an artery, rather than a vein, to ensure that the blood gasses accurately reflect the percentage and quantity of blood gasses that are in the blood after passing through the lungs. Once the blood enters veins and capillaries, oxygen is exchanged for carbon dioxide. A venous blood sample would not, therefore, accurately reflect the percentage and quantity of blood gasses that are in the blood after passing through the lungs. Accordingly, an arterial blood sample is utilized to provide a relatively accurate determination regarding blood gas data.

Unlike blood gas data, the general blood data remains relatively constant throughout the circulatory system. In theory, therefore, blood from either a venous blood sample or an arterial blood sample may be utilized to gain general blood data. That is, general blood data may be gained by analyzing blood samples that are collected from either a vein or an artery. Despite the fact that general blood data may be gained from blood collected from either a vein or an artery, phlebotomists generally collect (a) a venous blood sample to gain general blood data and (b) an arterial blood sample to gain blood gas data. Even when both general blood data and blood gas data are requested by the physician, phlebotomists conventionally collect both a venous blood sample and an arterial blood sample.

### SUMMARY OF THE INVENTION

One aspect of the invention involves an adapter for collecting a blood sample. The adapter includes a stopper and a housing. The stopper is formed of a material that is piercable by a needle and sealable upon withdrawal of the needle. The housing, which has a first end and an opposite second end, defines an aperture extending through the housing from the first end to the second end. The stopper is located in the aperture and adjacent the first end. In addition, the second end has a configuration that is joinable with a syringe.

Another aspect of the invention involves a kit for drawing blood. The kit includes a needle device, a holder, a syringe, and an adapter. The needle device has a first needle end and a second needle end. The holder is joinable with the needle device such that the first needle end extends outward from the holder and the second needle end extends into an interior area of the holder. The adapter has a first end and an opposite second end connected by an aperture extending through the adapter. The first end has dimensions that fit into the interior area of the holder, and the second end has a configuration that joins with the syringe. In addition, the adapter includes a stopper located in the aperture and adjacent the first end.

Yet another aspect of the invention involves a method of collecting a first blood sample and a second blood sample. The method includes joining an adapter to a syringe. An artery is punctured with a first needle end that is in fluid communication with a second needle end. A first blood sample is collected by piercing the stopper with the second needle end and depositing the first blood sample within the syringe. A second blood sample is collected by inserting the second needle end into a vacuum container and depositing the second blood sample within the vacuum container.

The advantages and features of novelty characterizing aspects of the invention are pointed out with particularity in the appended claims. To gain an improved understanding of the advantages and features of novelty, however, reference may be made to the following descriptive matter and accompanying drawings that describe and illustrate various embodiments and concepts related to aspects of the invention.

### DESCRIPTION OF THE DRAWINGS

The foregoing Summary of the Invention, as well as the following Detailed Description of the Invention, will be better understood when read in conjunction with the accompanying drawings.

FIG. 1 is a plan view of various elements of a prior art venous blood sample kit.

FIG. 2 is a plan view of various elements of a prior art arterial blood sample kit.

FIG. 3 is a plan view of an adapter in accordance with aspects of the present invention.

FIG. 4 is a cross-sectional view of the adapter, as defined by line 4-4 in FIG. 3.

FIG. 5 is a plan view of the adapter and selected elements from the venous blood sample kit and the arterial blood sample kit.

FIG. 6 depicts a first general step in a method of utilizing the adapter.

FIG. 7 depicts a second general step in the method of utilizing the adapter.

FIG. 8 depicts a third general step in the method of utilizing the adapter.

### DETAILED DESCRIPTION OF THE INVENTION

Introduction

The following discussion and accompanying figures disclose an adapter for use in collecting blood samples and a method for utilizing the adapter. As discussed in the Background of the Invention section above, phlebotomists conventionally collect both a venous blood sample and an arterial blood sample when general blood data and blood gas data are requested by the physician. In collecting a venous blood sample, a venous blood draw kit is utilized to draw blood from a vein of an individual. Similarly, an arterial blood draw kit is utilized to draw blood from an artery of the individual and collect an arterial blood sample. Accordingly, two separate blood draw kits are utilized, and the individual must endure two separate needle pricks when general blood data and blood gas data are requested by the physician.

The adapter, as discussed below, may be utilized in conjunction with a single blood draw kit to collect blood from an artery that is then utilized to gain both general blood data and blood gas data. An advantage of the adapter is that the overall biological waste (i.e., needles and other blood draw kit components) produced from collecting blood samples is reduced. The adapter also reduces the number of needle pricks endured by the individual, thereby decreasing the discomfort experienced by the individual. Additionally, the adapter reduces the time necessary to collect blood samples, which increases the efficiency of the phlebotomist and further decreases the discomfort of the individual. Accordingly, use of the adapter may impart various advantages over the conventional use of two separate blood draw kits when general blood data and blood gas data are requested by the physician.

Venous Blood Draw Kit

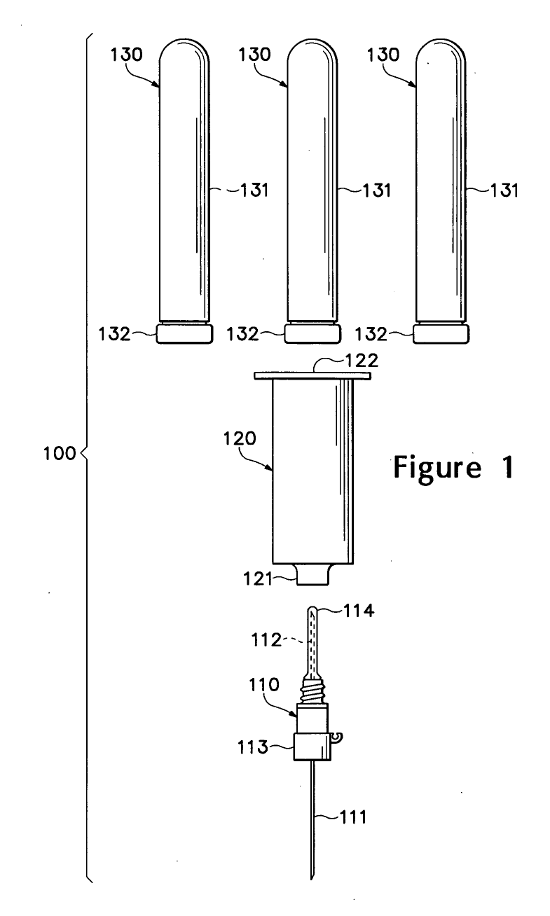

Prior to discussing the adapter in detail, the features and use of conventional blood draw kits will be explained. Relevant portions of a conventional venous blood draw kit 100 are depicted in FIG. 1 and include a dual needle device 110, a tube holder 120, and a plurality of vacuum containers 130. Venous blood draw kit 100 may also include additional elements that are not depicted or discussed further, such as a cap for portions of dual needle device 110.

The primary elements of dual needle device 110 are a collection needle 111, a tube needle 112, a joining element 113, and a sleeve 114. Collection needle 111 protrudes out of one side of joining element 113 and has a configuration that is suitable for entering a vein when collecting a venous blood sample. Tube needle 112 protrudes out of an opposite side of joining element 113 and has a configuration that is suitable for entering one or more of vacuum containers 130 when collecting a venous blood sample. Collection needle 111 and tube needle 112 are aligned and in fluid communication such that blood entering collection needle 111 may pass through tube needle 1 12. Although collection needle 111 and tube needle 112 are discussed herein as two separate needles, collection needle 111 and tube needle 112 may be one needle with two opposite ends that are sharpened. Joining element 113 is centrally located with respect to collection needle 111 and tube needle 112, and joining element 113 has a configuration that securely mates with tube holder 120. Sleeve 114 is formed from a rubber or latex material, for example, that extends over tube needle 112. As discussed below, tube needle 112 protrudes through or otherwise punctures sleeve 114 when entering one of vacuum containers 130. When tube needle 112 is withdrawn from one of vacuum containers 130, sleeve 114 again extends over tube needle 112 to cover tube needle 112.

Tube holder 120 has a generally cylindrical configuration that includes a connection end 121 and an opposite open end 122. Connection end 121 securely mates with joining element 113 to join dual needle device 110 and tube holder 120. Open end 122 is wider than connection end 121 and has an inside diameter that accepts one of vacuum containers 130. That is, one of vacuum containers 130 may extend into tube holder 120 through open end 122. Suitable materials for tube holder 120 include a variety of polymers.

Vacuum containers 130 each include a vial portion 131 and a stopper portion 132. Vial portion 131 has a generally cylindrical and elongate configuration that fits within tube holder 120 by extending through open end 122. One end of vial portion 131 is rounded and closed, and an opposite end is open. The interior of vial portion 131 holds at least a partial vacuum, and stopper portion 132 extends across the open end of vial portion 131 to seal the open end and prevent atmosphere from entering vial portion 131. Whereas vial portion 131 may be formed from a polymer or glass material, for example, stopper portion 132 may be formed from a rubber or latex material that tube needle 112 may puncture. More particularly, stopper portion 132 may be formed from any material that (a) will form a seal to limit fluid from entering vial portion 131, (b) is piercable with a needle (e.g., tube needle 112), and (c) will seal when the needle is withdrawn.

In taking a venous blood sample, dual needle device 110 is joined with tube holder 120.

In this configuration, collection needle 111 extends outward and away from tube holder 120, and tube needle 112 is axially located within tube holder 120 and extends toward open end 122. Collection needle 111 is then utilized to puncture a vein of the individual such that an end of collection needle 111 extends into the vein. At this stage, sleeve 114 extends over tube needle 112 and prevents blood from exiting tube needle 112 should the blood be pressurized. One of vacuum containers 130 is then placed in tube holder 120 such that tube needle 112 is adjacent stopper portion 132. When the phlebotomist intends to draw blood from the vein, vacuum container 130 is pressed against tube needle 112 such that an end of tube needle 112 extends through both of sleeve 114 and stopper portion 132 to enter the interior of vial portion 131. The vacuum within vial portion 131 forms a negative pressure with blood in the vein, and the blood is drawn through each of collection needle 111 and tube needle 112 so as to be deposited within vacuum container 130. Once vacuum container 130 holds a sufficient quantity of blood, vacuum container is removed from tube holder 120 such that tube needle 112 exits stopper portion 132. The rubber or latex material of stopper portion 132 then closes to seal the blood within vacuum container 130. The material of sleeve 114 also extends over tube needle 112 to prevent additional blood from exiting tube needle 112. If additional venous blood samples are required, one or more additional vacuum tubes 130 may be utilized in a similar manner. A laboratory technician then analyses the venous blood sample.

Each of vacuum containers 130 may contain an additive that is identified through a color-coded system. For example, stopper portion 132 may be red to indicate that no additive is present, light blue to indicate the presence of sodium citrate for coagulation studies, green to indicate the presence of heparin, lavender to indicate the presence of ethylenediaminetetraacetic acid, red and gray to indicate the presence of a clot activator, or orange to indicate the presence of thrombin. Accordingly, multiple vacuum containers that serve different purposes may be utilized when drawing blood from a single individual.

Arterial Blood Draw Kit

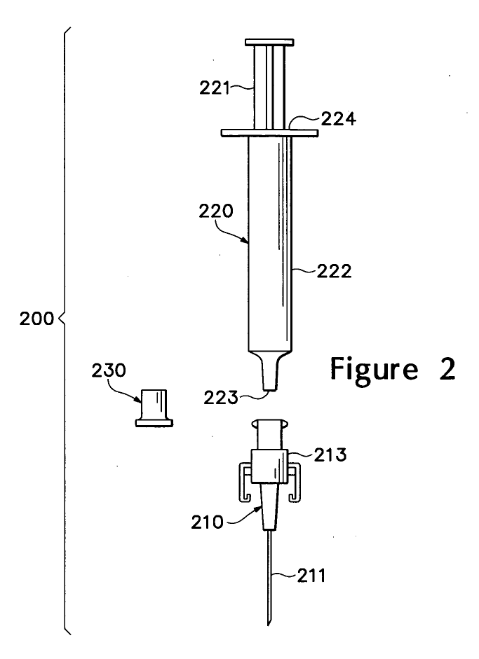

Relevant portions of a conventional arterial blood draw kit 200 are depicted in FIG. 2 and include a needle device 210, a syringe 220, and a cap 230. Arterial blood draw kit 200 may also include additional elements that are not depicted or discussed further, such as an additional cap for portions of needle device 210.

The primary elements of needle device 210 are an collection needle 211 and a joining element 213. Collection needle 211 protrudes out of one side of joining element 213 and has a configuration that is suitable for entering an artery when collecting an arterial blood sample. Joining element 213 is located at an end of collection needle 211 and has a configuration that securely mates with syringe 220. More particularly, joining element 213 is structured to incorporate a female portion of a Luer connection that interfaces with syringe 220.

Syringe 220 has a configuration that includes a plunger 221 and a body 222. Plunger 221 has an elongate configuration that extends into body 222 and moves axially along a length of body 222. Body 222 has a generally cylindrical configuration that defines a connection end 223 and an opposite open end 224. Connection end 223 is narrower than open end 224 and is structured to incorporate a male portion of a Luer connection that interfaces with joining element 213. That is, the male portion of the Luer connection (i.e., connection end 223) extends into the female portion of the Luer connection (i.e., joining element 213) to join syringe 220 to needle device 210. In addition, cap 230 has a structure that may extend into or around connection end 223.

In taking an arterial blood sample, needle device 210 is joined with syringe 220. In this configuration, collection needle 211 extends outward and away from syringe 220.

Collection needle 211 is then utilized to puncture an artery of the individual such that an end of collection needle 211 extends into the artery. When the phlebotomist intends to draw blood from the artery, plunger 221 is pulled in a direction that is away from needle device 210 and toward open end 224. This action effectively forms a negative pressure within syringe 220, and the blood is drawn through collection needle 211 so as to be deposited within body 222. Once syringe 220 holds a sufficient quantity of blood, collection needle 211 is withdrawn from the artery, syringe 220 is separated from needle device 210, and cap 230 is placed into connection end 223 to seal the blood within syringe 220. Following collection of the arterial blood sample, a laboratory technician then analyses the arterial blood sample. More particularly, cap 230 is removed and connection end 223, which has the male portion of the Luer connection, is joined with a blood gas analyzer, which has another female portion of the Luer connection in order to facilitate joining syringe 220 with the analyzer.

Adapter

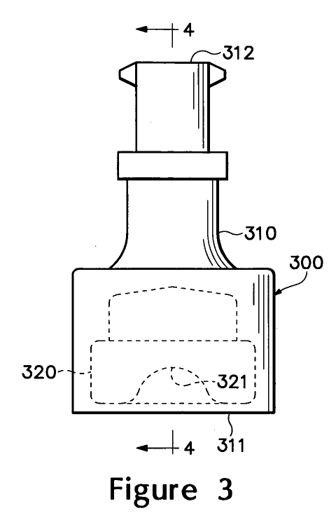

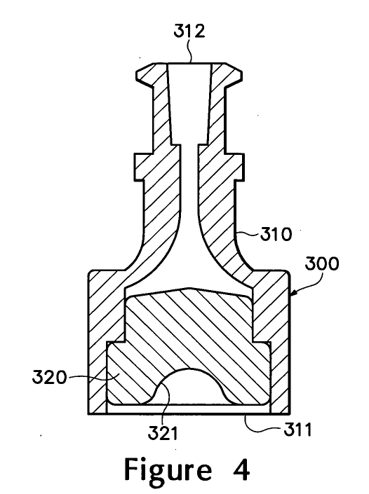

Despite the fact that general blood data may be gained from blood collected from either a vein or an artery, phlebotomists generally utilize (a) a blood draw kit similar to venous blood draw kit 100 to collect a venous blood sample and gain general blood data and (b) a blood draw kit similar to arterial blood draw kit 200 to collect an arterial blood sample and gain blood gas data. Even when both general blood data and blood gas data are requested by the physician, phlebotomists conventionally collect both a venous blood sample and an arterial blood sample. This procedure effectively requires that the phlebotomist perform two separate blood collection procedures in order to gain general blood data and blood gas data. An adapter 300, which is depicted individually in FIGS. 3 and 4, permits a phlebotomist to draw blood from an artery that may be utilized to gain general blood data and blood gas data. That is, adapter 300 permits the phlebotomist to perform only one procedure in order to gain general blood data and blood gas data.

As described in greater detail below, adapter 300 is utilized in conjunction with various elements from venous blood draw kit 100 and arterial blood draw kit 200 in order to draw a blood sample from an artery. In addition to collecting blood in a syringe, such as syringe 220, the blood drawn from the artery may also be collected in one or more vacuum containers, such as vacuum containers 130. As discussed above, adapter 300 permits the phlebotomist to perform only one procedure in order to gain general blood data and blood gas data. Further advantages to the use of adapter 300 include reducing the number of needle pricks endured by the individual, reducing the time necessary to collect blood samples, decreasing the discomfort experienced by the individual, and reducing the overall biological waste produced from collecting blood samples.

The primary elements of adapter 300 are a housing 310 and a stopper 320. Housing 310 is formed from a polymer material, for example, and has a generally hollow configuration that includes a stopper end 311 and an opposite connection end 312. In effect, an aperture extends through housing 310 and between ends 311 and 312. The shape, dimensions, and overall configuration of housing 310 may vary significantly. In general, however, stopper end 311 is configured to receive a structure that limits the passage of fluids (e.g., stopper 320), and connection end 312 interfaces with a syringe (e.g., syringe 220).

Stopper end 311 has an exterior diameter that approximates the diameter of vacuum containers 130. As with vacuum containers 130, therefore, stopper end 311 will fit into tube holder 120. Connection end 312 has a configuration that is narrower than stopper end 311 and is structured to incorporate a female portion of the Luer connection. That is, connection end 312 has a configuration that interfaces with syringe 220 and, more particularly, with connection end 223 of syringe 220. Housing 310 is depicted as having a configuration that steps down in diameter between stopper end 311 and connection end 312. In some aspects of the invention, for example, the diameter may taper between stopper end 311 and connection end 312, or the diameter may remain constant between stopper end 311 and connection end 312. Housing 310 is also depicted as being formed from a single, unitary element of material that defines stopper end 311, connection end 312, and the aperture that extends through housing 310. In other aspects of the invention, however, housing 310 may be formed from multiple elements that are joined to form the general structure discussed above.

Based upon the above discussion, housing 310 exhibits various dimensions that facilitate aspects of the invention. In general, the exterior dimensions of stopper end 311 are selected to fit within tube holder 120, and the interior dimensions of stopper end 311 are selected to accommodate stopper 320. The primary purpose of connection end 312 is to join with syringe 220, but the dimensions and configuration of connection end 312 may vary significantly. Accordingly, connection end 312 may have any dimensions or configurations that join with syringe 220. As discussed above, syringe 220 incorporates a male portion of a Luer connection. In this situation, the interior dimensions of connection end 312 are selected to receive or otherwise join with syringe 220, and may have a configuration of a female portion of the Luer connection. As depicted, the exterior dimensions of connection end 312 are less than the exterior dimensions of stopper end 311, but may be the same or greater. Accordingly, the specific dimension and configuration of housing 310 may vary significantly.

Stopper 320 is located within stopper end 311 and effectively prevents fluids, such as blood and atmosphere, from passing through adapter 300. When a needle punctures and extends through stopper 320, however, blood may flow from stopper end 311 to connection end 312, where the blood is collected in a syringe. Stopper 320 is formed of a material that is similar to the materials forming stopper portion 132 of vacuum containers 130. That is, stopper 320 may be formed from a rubber or latex material and, more particularly, any material that (a) will form a seal to limit fluid from entering or passing through adapter 300, (b) is piercable with a needle (e.g., tube needle 112), and (c) will seal when the needle is withdrawn. Stopper 320 has a generally cylindrical configuration, and edge surfaces of stopper 320 contact the inside surface of housing 310.

In this configuration, stopper 320 presses against the inside surface and effectively prevents fluids from passing through adapter 300, but a needle may pass through stopper 320 when collecting blood. In order to decrease the force necessary to pass a needle through stopper 320, a central area of stopper 320 may define an indentation 321 that reduces the overall thickness in the area where the needle will puncture stopper 320.

Stopper 320 is discussed above and depicted in the figures as having a generally cylindrical configuration that fits within stopper end 311. As a comparison, therefore, stopper 320 may have the general shape of stopper portions 132 of vacuum tubes 130.

This provides an example of a configuration that is suitable for stopper 320. Any structure, however, that limits the passage of fluids until punctured by a needle may be utilized for stopper 320. Accordingly, a variety of valve structures and membranes, for example, may be suitable for stopper 320.

As noted above, stopper 320 effectively prevents fluids, such as blood and atmosphere, from passing through adapter 300 until punctured by a needle. Even when punctured by a needle, however, stopper 320 continues to prevent fluids from passing through adapter 300 unless the fluids first pass through the needle. Once the needle is retracted from stopper 320 and removed from adapter 300, the rubber or latex material of stopper 320 then closes to prevent atmosphere, for example, from engaging the blood. Closing of the rubber or latex material also prevents the blood from passing back through adapter 300 in a reverse direction. Accordingly, stopper 320 effectively seals the blood in the syringe and prevents the blood from escaping the syringe.

Use of the Adapter

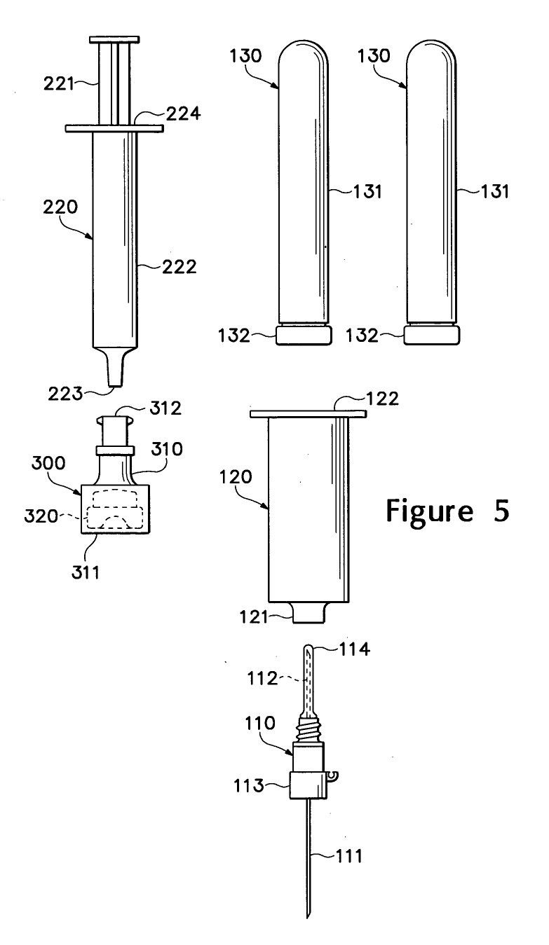

With reference to FIG. 5, adapter 300 is shown in combination with various elements from venous blood draw kit 100 and arterial blood draw kit 200. More particularly, adapter 300 is shown with dual needle device 110, tube holder 120, multiple vacuum containers 130, and syringe 220. Adapter 300 and these various elements from venous blood draw kit 100 and arterial blood draw kit 200 may be utilized to collect multiple arterial samples in order to gain general blood data and blood gas data. That is, blood drawn from an artery may be collected in each of the multiple vacuum containers 130 and syringe 220.

As discussed above, phlebotomists conventionally collect both a venous blood sample and an arterial blood sample when a physician requests both general blood data and blood gas data. A laboratory technician then analyses the blood samples on specialized equipment that is configured to gain (a) general blood data from blood collected in vacuum containers 130 and (b) blood gas data from blood collected in syringe 220. The use of adapter 300 does not vary the manner in which the laboratory technician analyzes the blood samples since the phlebotomist continues to collect blood in multiple vacuum containers 130 and syringe 220. Rather, the use of adapter 300 only changes the method performed by the phlebotomist in collecting the blood samples.

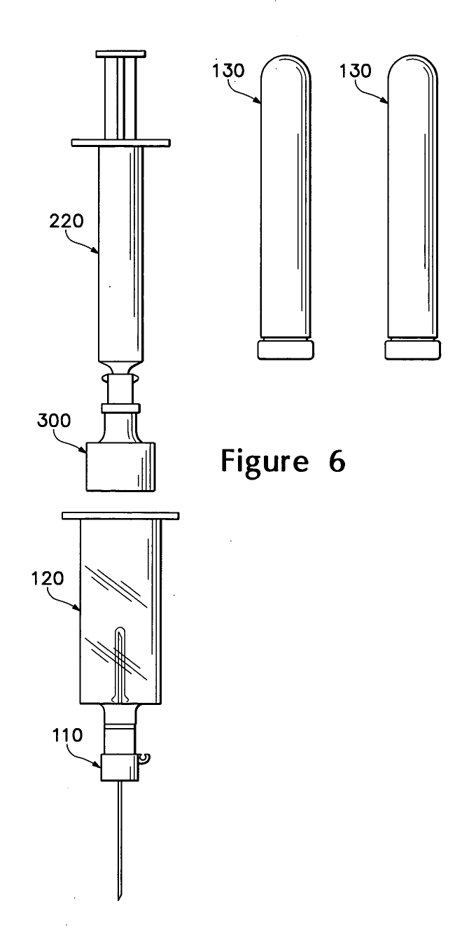

The method performed by the phlebotomist in collecting blood samples with adapter 300 will now be discussed in detail with reference to FIGS. 6-8. The method discussed below is intended to provide an example of the general steps that the phlebotomist will perform in collecting blood samples with adapter 300. One skilled in the relevant art will recognize, however, that these general steps may be modified or additional steps may be performed without departing from the scope of the invention. Accordingly, the method discussed below is merely an example of the manner in which adapter 300 may be used by the phlebotomist.

FIG. 6 depicts a first general step in the method of utilizing adapter 300 to collect blood samples. In the first general step, dual needle device 110 is joined with tube holder 120, adapter 300 is joined with syringe 220, and multiple vacuum containers 130 are present. As discussed above, joining element 113 of dual needle device 110 has a configuration that securely mates with connection end 121 of tube holder 120. In this configuration, collection needle 111 extends outward and away from tube holder 120, and tube needle 112 is axially located within tube holder 120 and extends toward open end 122 of tube holder 120. Connection end 312 of adapter 300 is structured to incorporate a female portion of the Luer connection. Correspondingly, connection end 223 of syringe 220 is structured to incorporate a male portion of the Luer connection. As depicted in FIG. 6, therefore, adapter 300 is configured to join with syringe 220 by uniting connection ends 312 and 223.

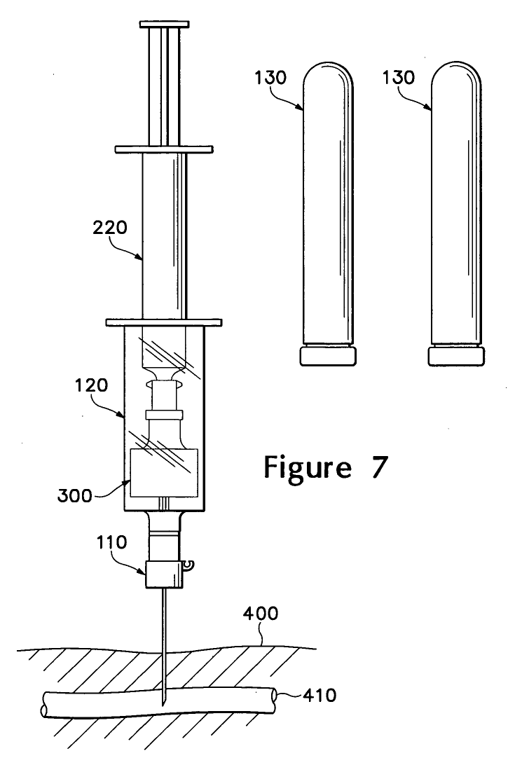

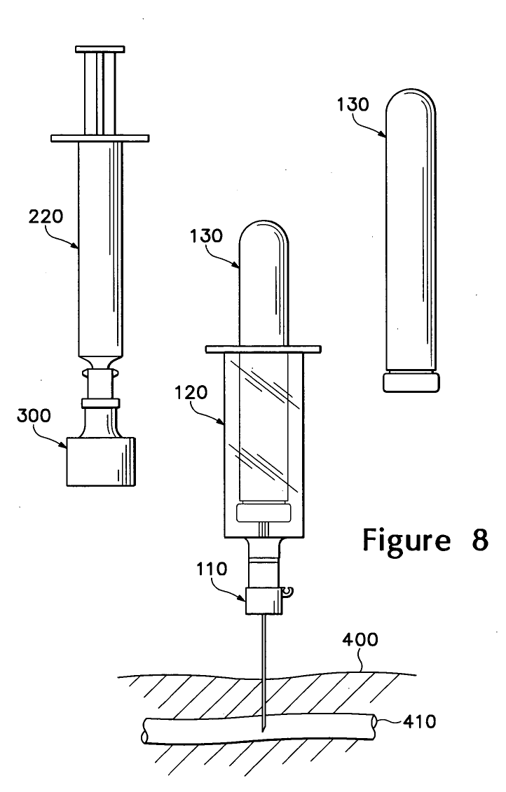

FIG. 7 depicts a second general step in the method of utilizing adapter 300 to collect blood samples. In the second general step, collection needle 111 is utilized to pierce an individual (represented by reference numeral 400 in FIGS. 7 and 8) and enter an artery (represented by reference numeral 410 in FIGS. 7 and 8) of the individual to collect a blood sample in syringe 220. When adapter 300 is joined with syringe 220, stopper 320 is exposed. As discussed above, stopper end 311 of adapter 300 will fit into tube holder 120 because stopper end 311 has a diameter that approximates the diameter of vacuum containers 130. In addition, stopper 320 effectively prevents fluids from passing through adapter 300, but a needle may pass through stopper 320 when collecting blood. Accordingly, adapter 300 may extend into tube holder 120 such that an end of tube needle 112 pierces stopper 320 and passes through stopper 320. The phlebotomist then moves plunger 221 rearward to draw blood from the artery into syringe 220. More particularly, moving plunger 221 rearward draws blood through each of collection needle 111 and tube needle 112 in order to collect the blood in body 222 of syringe 220.

When tube needle 112 extends through stopper 320, an end of tube needle 112 is located in the hollow area within housing 310 of adapter 300. The hollow area (i.e., aperture) within housing 310 is adjacent to connection end 223 of syringe 220, and blood that enters the hollow area within housing 310 may freely flow into syringe 220. Accordingly, blood that passes through collection needle 111 and tube needle 112 is collected in syringe 220.

FIG. 8 depicts a third general step in the method of utilizing adapter 300 to collect blood samples. In the third general step, adapter 300 is withdrawn from tube holder 120 and further samples of blood are collected in vacuum containers 130. Once adapter 300 is removed from tube holder 120, one of vacuum containers 130 may be placed within tube holder 120 such that an end of tube needle 112 extends through stopper portion 132 and blood is collected in vial portion 131. Once a sufficient quantity of blood is collected, this vacuum container 130 may be withdrawn from tube holder 120 and one or more additional vacuum containers 130 may be utilized in a similar manner to collect additional blood samples. Once all blood samples are collected, collection needle 111 may be withdrawn from the artery to effectively complete the method.

Additional considerations relating to the method of utilizing adapter 300 to collect blood samples will now be discussed. Sleeve 114 extends over tube needle 112 and may be pierced by tube needle 112. When tube needle 112 extends through stopper 320 or one of stopper portions 132, sleeve 114 retracts to a base of tube needle 112. When tube needle 112 is withdrawn from stopper 320 or one of stopper portions 132, however, sleeve 114 again extends over tube needle 112 and prevents further blood from exiting tube needle 112. The material that forms sleeve 114 is resilient and effectively forms a seal over tube needle 112. A pressure of the blood within the artery is elevated in relation to atmosphere outside of sleeve 114. Despite this difference in pressure between the blood within the artery and the atmosphere outside of sleeve 114, the seal of sleeve 114 is generally sufficient to prevent the pressurized blood within the artery from escaping.

When adapter 300 is withdrawn from tube holder 120, adapter 300 is joined with syringe 220 and stopper 320 forms an effective seal that prevents atmosphere from modifying the blood gas content of the blood sample. Adapter 300 may, therefore, remain joined with syringe 220 while the blood sample is transported to the laboratory technician for analysis in order to maintain the analytical integrity of the blood sample. Accordingly, an advantage of the use of adapter 300 over the conventional method of collecting an arterial blood sample is that blood gas data may be more accurate. As an alternative, however, the phlebotomist may disconnect adapter 300 from syringe 220 and a cap (i.e., cap 230) may be placed into connection end 223 of syringe 220.

The method discussed above effectively involved a single blood collection procedure. That is, the phlebotomist drew all required blood samples from an artery, rather than an artery and a vein. Accordingly, the use of adapter 300 has the advantages of reducing the number of needle pricks endured by the individual, decreasing the discomfort experienced by the individual, and reducing the time necessary to collect blood samples.

In addition, the method simplifies the overall procedure for collecting blood samples that are utilized to gain general blood data and blood gas data.

A further advantage to the use of adapter 300 relates to the existing equipment that a laboratory technician utilizes to analyze blood samples. Blood analysis equipment is configured to remove blood from vacuum containers 130 when general blood data is required, and the blood analysis equipment is configured to remove blood from syringe 220 when blood gas data is required. The method described above collects blood samples within both vacuum containers 130 and syringe 220. Accordingly, the laboratory technician may utilize existing equipment and procedures to analyze the blood samples.

Yet another advantage to the use of adapter 300 relates to a lesser probability that the phlebotomist will be accidentally pricked by a needle. When needle device 210 is being separated from syringe 220 or when collection needle 211 is being withdrawn from the individual, the fingers of the phlebotomist are placed in close proximity to collection needle 211. Although relatively rare, the fingers may be pricked or otherwise punctured by collection needle 211, which may expose the phlebotomist to blood-borne pathogens. Adapter 300 permits syringe 220 to be separated from dual needle device 110 and set aside. The phlebotomist is then free to use both hands when withdrawing dual needle device 110 from the individual, thereby lessening the probability that the phlebotomist will be accidentally pricked by a needle.

Packaging of the Adapter

Adapter 300 may be packaged individually for use by the phlebotomist. In situations where the physician requests general blood data and blood gas data, the phlebotomist may obtain (a) a first package containing venous blood draw kit 100, (b) a second package containing arterial blood draw kit 200, and (c) a third package containing adapter 300. The phlebotomist will then utilize adapter 300 and the various elements from venous blood draw kit 100 and arterial blood draw kit 200 in order to draw blood from an artery for use in providing the physician with general blood data and blood gas data. That is, the phlebotomist will then utilize adapter 300 in combination with dual needle device 110, tube holder 120, one or more vacuum containers 130, and syringe 220 from blood draw kits 100 and 200. Those portions of venous blood draw kit 100 and arterial blood draw kit 200 that are not utilized to draw blood from the artery are then discarded.

As an alternative, adapter 300 may be packaged with the various elements from venous blood draw kit 100 and arterial blood draw kit 200 that are utilized in order to draw blood from an artery. That is, adapter 300 may be packaged with dual needle device 110, tube holder 120, one or more vacuum containers 130, and syringe 220. When drawing blood from the artery, therefore, each of these elements are utilized. In addition to increasing the simplicity of drawing blood from the artery, this manner of packaging reduces the overall biological waste produced from collecting blood samples. More particularly, other elements of venous blood draw kit 100 and arterial blood draw kit 200, which are not utilized and include needle device 210, are not present in the packaging with adapter 300 and do not form additional biological waste.

### CONCLUSION

The above discussion and accompanying figures disclose adapter 300 as being used in conjunction with elements from a conventional venous blood draw kit 100 and a conventional arterial blood draw kit 200. Although actual elements from conventional kits may be utilized, elements that are specially-designed to be utilized with adapter 300 may also be utilized. Furthermore, the above discussion and accompanying figures disclose the structure of adapter 300 and the use of adapter 300 in collecting blood samples from an individual (i.e., a human). Although adapter 300 is discussed above in the context of drawing blood from a human, adapter 300 may also be utilized in the context of veterinary medicine to collect blood samples from animals.

Aspects of the present invention are disclosed above and in the accompanying drawings with reference to a variety of embodiments. The purpose served by the disclosure, however, is to provide an example of the various features and concepts related to aspects of the invention, not to limit the scope of the invention. One skilled in the relevant art will recognize that numerous variations and modifications may be made to the embodiments described above without departing from the scope of the present invention, as defined by the appended claims.

1. An adapter for collecting a blood sample, the adapter comprising:
a housing having a first end and an opposite second end, the housing defining an aperture extending through the housing from the first end to the second end, the first end having a first interior dimension extending across the aperture, and the second end having a second interior dimension extending across the aperture, the first interior dimension being greater than the second interior dimension; and
a stopper located within the aperture and adjacent the first end of the housing, the stopper forming a seal that limits fluids from passing through the aperture, and the stopper being formed of a material that is piercable by a needle.

2. The adapter recited in claim 1, wherein the second end of the housing incorporates a connector for joining the adapter to a syringe.

3. The adapter recited in claim 2, wherein the connector is formed of unitary construction with the housing.

4. The adapter recited in claim 1, wherein the first end has a first exterior dimension and the second end has a second exterior dimension, the first exterior dimension being greater than the second exterior dimension.

5. The adapter recited in claim 4, wherein the first exterior dimension is selected to be less then a diameter of a tube holder.

6. The adapter recited in claim 1, wherein the stopper defines an indentation oriented to extend into the aperture from the first end.

7. An adapter for collecting a blood sample, the adapter consisting of:
a stopper formed of a material that is piercable by a needle and sealable upon withdrawal of the needle; and
a housing having a first end and an opposite second end, the housing defining an aperture extending through the housing from the first end to the second end, the stopper being located in the aperture and adjacent the first end, and the second end having a connector that is joinable with a syringe.

8. The adapter recited in claim 7, wherein the first end has a first interior dimension extending across the aperture, and the second end has a second interior dimension extending across the aperture, the first interior dimension being greater than the second interior dimension.

9. The adapter recited in claim 7, wherein the stopper forms a seal with a surface of the aperture that limits fluids from passing through the aperture

10. The adapter recited in claim 7, wherein exterior dimensions of the first end are selected to be less than a diameter of a tube holder.

11. The adapter recited in claim 7, wherein the stopper defines an indentation oriented to extend into the aperture from the first end.

12. A kit for drawing blood, the kit comprising:
a needle device having a first needle end and a second needle end that are in fluid communication;
a holder that is joinable with the needle device such that the first needle end extends outward from the holder and the second needle end extends into an interior area of the holder;
a syringe; and
an adapter having a first end and an opposite second end connected by an aperture extending through the adapter, the first end having dimensions that fit into the interior area of the holder, and the second end having a configuration that joins with the syringe, and the adapter including a stopper located in the aperture and adjacent the first end.

13. The kit recited in claim 12, wherein the needle, the holder, the syringe, and the adapter are contained within a single package.

14. The kit recited in claim 12, further including at least one vacuum container.

15. The kit recited in claim 12, wherein the stopper forms a seal that limits fluids from passing through the aperture, and the stopper is formed of a material that is piercable by a needle.

16. The kit recited in claim 12, wherein each of the syringe and the second end of the adapter include corresponding portions of a Luer connector.

17. The kit recited in claim 12, wherein the dimensions of the first end are greater than dimensions of the second end.

18. The kit recited in claim 12, wherein the stopper defines an indentation oriented to extend into the aperture from the first end.

19. A method of collecting a first blood sample and a second blood sample, the method comprising steps of:
joining an adapter to a syringe, the adapter having a hollow configuration that defines a first end and an opposite second end, the first end including a stopper and the second end being configured to join with the syringe;
puncturing an artery with a first needle end that is in fluid communication with a second needle end;
collecting a first blood sample by piercing the stopper with the second needle end and depositing the first blood sample within the syringe; and
collecting a second blood sample by inserting the second needle end into a vacuum container and depositing the second blood sample within the vacuum container.

20. The method recited in claim 20, further including a step of analyzing the first blood sample and the second blood sample.
